(19) United States
(12) Patent Application Publication
(10) Pub. No.: US 2020/0087616 A1
(43) Pub. Date: Mar. 19, 2020

(54) CELL SUPPORTING SERUM

(71) Applicant: SERUCELL CORPORATION, Palo Alto, CA (US)

(72) Inventors: Cortland Bohacek, Woodside, CA (US); Walter De Paula Neto, Lavalette, WV (US)

(21) Appl. No.: 16/570,472

(22) Filed: Sep. 13, 2019

Related U.S. Application Data
(60) Provisional application No. 62/731,758, filed on Sep. 14, 2018.

(51) Int. Cl. C12N 5/00

(52) U.S. Cl. CPC C12N 5/0018; C12N 2501/10; C12N 2502/50

(57) ABSTRACT

Failing and degenerating organs may be induced to recover by administering a serum augmenting natural paracrine to extend replenishment. The serum may be collected from a stressed culture of cells and administered to an organ and/or tissue. The stressed serum may also be combined with cells to be implanted to increase recovery, viability and/or establishment of cells.

### FIELD

A serum supporting the replenishment of tissues and/organs, as well as the viability, recovery and/or establishment of implanted cells, and methods of increasing replenishment of tissues and/organs, as well as the viability, recovery and/or establishment of implanted cells.

### BACKGROUND

Organs and tissues of the body can sustain serious damage from a variety of common occurrences. A stroke or heart attack, for instance, may cause serious damage to a patient's heart. Diabetes and hypertension may cause serious damage to the kidneys. The damage caused by these common conditions may lead to organ failure. While transplant is an option, many patients are not suitable recipients. Those that are, such as children having degenerative diseases, may not receive a suitable transplant in time.

Additionally, some medical conditions afflicting children or other individuals are not curable by transplants. Children suffering degenerative disorders, such as multiple sclerosis, are inflected with a failing nervous system. Unfortunately, replacing damaged and degenerating nerves with transplants is not yet possible.

Treatment of cancer in children may also cause serious degenerative conditions. For instance, children being treated for leukemia may develop graft-versus-host disease. Chemotherapy may also cause a child to develop foot-hand syndrome, leading to painful sensations in the feet or hands that reduce mobility, play and the ability to be child.

Stem cells have received significant attention as potential alternatives for organ transplant, treatments for degenerative disease and treatments of for side effects of cancer therapy. Despite promising results using experimental animals, clinical use of stem cells has remained elusive. The clinical use of stem cells requires a system that is feasible for use in hospital and clinics. Unlike laboratories conducting animal studies, many hospitals and clinics lack the means for maintaining stem cell lines. As such, stem cells utilized in hospital and clinical settings are often received frozen from a supplier. At the time of treatment, the cryopreserved stem cells are thawed and implanted into the patient.

Unfortunately, stem cells struggle to recover and establish themselves after cryopreservation. Struggling to recover, cryopreserved stem cells are not as effective as fresh stem cells utilized in research laboratories, often making the cells less effective in clinical use. As such, the clinical use of stem cells has not been able to replicate the promising results obtained in animal studies.

As an alternative to stem cell therapy, isolation and reintroduction of a patient's cells, or that of a donor, have been attempt to repair degenerating and damaged tissues and organs. The efficacy of these attempts has been limited by the ability of the implanted cells to establish themselves after transplantation.

Attempting to avoid the inability of transplanted and reintroduced cells to establish themselves, growth factor therapy has been attempted with limited efficacy.

### SUMMARY

Recovery of failing, damaged and/or degenerating organs and/or tissue may be induced by administering a serum facilitating replenishment. Replenishment is the natural process by which tissues and/or organs replace cells, maintain integrity, receive nutrients, respond to changes and/or recover from stress. Replenishment of organs and/or tissues is controlled by paracrine signaling. Augmenting natural paracrine as to supplement, amplify and/or otherwise extend natural replenishment, accordingly, may increase recovery of failing, damaged and/or degenerative organs and/or tissues. Augmented paracrine signaling increasing replenishment of organs and/or tissue may include angio-modifications, morphogenesis of cells, changes to the formulation of the extracellular matrix, modifications of the extracellular matrix, activation of the immune system and modifications to cytoskeletons. Such augmented paracrine signaling may increase establishment of new cells, recovery of cells and/or growth of new cells by changing the tissue and/or cells in manner that may increase at least one of growth, migration and/or differential. Paracrine signaling inducing replenishment, rather than death, apoptosis and/or necrosis, may be triggered by subjecting cells to a recoverable stress. A serum collected from a stressed culture of cells, accordingly, may provide the various proteins, cytokines, glycans, hormones and other molecular factors necessary for improving replenishment. Such a stressed serum may be administered to an organ and/or tissue. The stressed serum may also be combined with donor cells, cells extracted from the patient and/or stem cells to increase at least one of recovery, viability and establishment.

The stressed serum may comprise a serum collected from a stressed cell culture and/or a synthetic serum produced from protein formulations. A serum collected from a stressed cell culture may comprise proteins, cytokines, glycans, hormones and other molecular factors extending replenishment to increase at least one of recovery, viability and establishment. Likewise, a serum formulated to simulate a serum collected from a stressed culture of cells may also extend replenishment. The stressed culture from which the serum is collected may comprise stem cells, one or more cells of the organ and/or tissue to be treated, one or more cells from a tissue and/or organ other than organ to be treated, and/or any combination therefore.

The stressed serum may be collected from a stressed culture of cells. Cells within a culture may be stressed by depriving the cells of at least one growth factor, nutrient and/or metabolic component. For example, cells within a culture may be stressed by depriving the cells of one or more growth factors while maintaining nutrient levels. The cells within the culture may be stressed at any time before reaching 100% confluence. For instance, cells within the culture may by stressed after the culture has grown to approximately 80 to 95% confluence or at other times. Accordingly, production of a stressed serum may be induced in a culture by replacing a growth medium inducing proliferation of the cells within the culture with a collection medium lacking all or a portion of the growth factors of the growth medium, after the culture has been grown to less than one-hundred percent confluence. For instance, the growth medium may be replaced with a collection medium lacking at least one of the growth factors of the growth medium. The culture may then be maintained for a period of time in the collection medium sufficient to allow the culture to produce from the collection medium a conditioned medium comprising the stressed serum. After which, at least a portion of the conditioned medium containing the stressed serum may be collected.

The stressed serum may be administered to organs and/or tissue by injecting a solution containing a sufficient amount of the stressed serum into the organ and/or tissue, adjacent to the damaged organ and/or tissue, and/or systemically. A sufficient amount refers to an amount of stressed serum extending replenishment by increasing at least one establishment, recovery, viability, growth, migration and/or differential of cells.

The stressed serum may also be administered using grafts containing a sufficient amount of the stressed serum, such as, but not limited to, skin grafts, vascular grafts, and/or grafts used to repair tissue damage in various organs such as the heart, liver, pancreas, nervous system, etc. Grafts containing the stressed serum ideally will be composed of biocompatible and/or bioabsorbable, such as poly(hydroxyvalerate), poly(L-lactic acid), polcaprolactone, poly(lactide-co-glycolide), poly(hydroxybutyrate), poly(hydroxybutyrate-co-valerate), polydioxanone, polyorthoesters, polyanhydrides, poly(glycolic acid), poly(D.L-lactic acid), poly(glycolic acid-co-trimethylene carbonate), polyphosphoesters, polyphosphoester urethanes, poly(amino acids), cyanoacrylates, poly(trimethylene carbonate), poly(iminocarbonate), copoly(ether-esters) (e.g. PEO/PLA), polyalkylene oxalates, polyphosphaZenes and biomolecules such as fibrin, fibrinogen, cellulose, starch, collagen, hyaluronic acid, etc., and mixtures thereof.

Matrixes containing the stressed serum may also be utilized to administer the stressed serum to an organ and/or tissue. Administering the serum with a matrix may be accomplished by placing the matrix within, on and/or near the organ and/or tissue to be treated. For instance, a matrix containing a sufficient amount of the stressed serum may be placed adjacent or otherwise in proximity to an organ to be treated. The stressed serum may also be incorporated into a hydrogel placed within, on and/or near the organ and/or tissue to be treated. Matrixes other than hydrogels may also be used. For instance, the stressed serum may be incorporated into a bandage placed over wound. The matrix should be sufficiently porous and/or have sufficient internal spaces as to hold and elude a sufficient amount of the stressed serum to extend replenishment. The matrix may be bioabsorbable as to eliminate the need for subsequent surgeries to retrieve the matrix. Additionally, absorption of the matrix by the body may facilitate release of a sufficient amount of the stressed serum over time. Appropriate bioabsorbable matrixes may be fabricated from poly(hydroxyvalerate), poly(L-lactic acid), polcaprolactone, poly(lactide-co-glycolide), poly(hydroxybutyrate), poly(hydroxybutyrate-co-valerate), polydioxanone, polyorthoesters, polyanhydrides, poly(glycolic acid), poly(D.L-lactic acid), poly(glycolic acid-co-trimethylene carbonate), polyphosphoesters, polyphosphoester urethanes, poly(amino acids), cyanoacrylates, poly(trimethylene carbonate), poly(iminocarbonate), copoly(ether-esters) (e.g. PEO/PLA), polyalkylene oxalates, polyphosphaZenes and biomolecules such as fibrin, fibrinogen, cellulose, starch, collagen, hyaluronic acid, etc., and mixtures thereof.

A sufficient amount of the stressed serum may also be incorporated into a spray formulation that can be applied onto tissue and/or organ to be treated.

A sufficient amount of the stressed serum may also be combined with cells extracted from the patients, donor cells and/or stem cells. Stem cells combined with the stressed serum may include, but are not limited to, mesenchymal stem cells, embryonic stem cells and/or induced pluripotent cells, to extend replenishment of implanted stem cells. When combined with cells, a sufficient amount refers to amount sufficient to increase recovery, viability and/or establishment of the cells.

When cryopreserved cells are to be used, the stressed serum may be combined with the cells prior to cryopreservation, during thawing, prior to implantation, during implantation and/or after implantation. When combined prior to cryopreservation, an amount of the stressed serum sufficient to increase recovery from cryopreservation and/or establishment of the cells may be combined with a cryopreservation solution. The stem cells may then be cryopreserved. Prior to implantation, the cryopreserved cells and stressed serum may be thawed. If the cryopreservation solution would elicit unwanted side effects if implanted, the cryopreservation solution may be separated from the stem cells and replaced with fresh a solution containing a sufficient amount of the stressed serum to facilitate increased establishment. Otherwise, the cells may be implanted with the cryopreservation solution and stressed serum. Regardless of if the stressed serum present during cryopreservation is removed, the presence of the stressed serum during thawing may facilitate subsequent recovery from cryopreservation, viability and/or establishment during subsequent implantation.

The stressed serum may also be used to facilitate recovery from cryopreservation, viability and/or establishment of cells when not part of a cryopreservation solution. For instance, cryopreserved stem cells may be combined with the stressed serum by thawing the cells in a solution containing a sufficient amount of the stressed serum to increase recovery, viability and/or establishment. After the stem cells have thawed, the cryopreservation solution and stressed serum solution may be removed. The removed stress serum may be replaced with a fresh solution containing the stressed serum and/or other solutions appropriate for the intended manner of implantation.

The cells may be implanted in a variety of manners, such as but not limited to, being overlaid onto the target tissue and/or organ, sprayed onto the target tissue and/or organ, deposited within or near the target tissue and/or organ, injected within or near the target tissue and/or organ, and/or systemically injected. The cells may be combined with a stressed serum at various times. For instance, the stressed serum may be combined with the cells prior to cryopreservation, when the cells are thawed, prior to implantation and/or after implantation. Derived from a culture of stressed cells, the stressed serum may contain various proteins, cytokines, glycans, hormones and/or other molecular factors of paracrine signaling inducing proliferation, growth and/or differentiation, as to induce the implanted stem cells to recover from cryopreservation, remain viable and/or establish themselves after implantation.

Depending on how the cells are to be implanted, the cells and stressed serum may be combined in various ways during and/or after implantation. For instance, if the cells are to facilitate regrowth of bone utilizing a bone matrix and/or graft, the cells and a sufficient amount of stressed serum may be incorporated into the graft and/or matrix. In such instance, the graft and/or matrix should be sufficiently porous and/or absorptive to hold a sufficient amount of the therapeutic serum increasing recovery from cryopreservation, viability and/or establishment. Cells may be incorporated into the matrix and/or graft before cryopreservation. In such instances, the matrix and/or graft may be infused with a cryopreservation solution and cryopreserved. The cryopreservation solution may include a sufficient amount of the stressed serum to increase recovery, viability and/or establishment of the cells. Alternatively, when the cryopreservation solution within the graft and/or matrix lacks the stressed serum, the matrix and/or graft may be thawed within a solution containing a sufficient amount of the stressed serum to increase recovery, viability and/or establishment of the cells.

Matrixes and/or grafts containing cells and a sufficient amount of the stressed serum to increase establishment and/or recovery may be used for grafts other than bone grafts, such as, but not limited to, skin grafts, vascular grafts, and/or grafts used to repair tissue damage in various organs such as the heart, liver, pancreas, nervous system, etc. Matrixes and/or grafts containing cells and the stressed serum ideally will be composed of biocompatible and/or bioabsorbable, such as poly(hydroxyvalerate), poly(L-lactic acid), polcaprolactone, poly(lactide-co-glycolide), poly(hydroxybutyrate), poly(hydroxybutyrate-co-valerate), polydioxanone, polyorthoesters, polyanhydrides, poly(glycolic acid), poly(D.L-lactic acid), poly(glycolic acid-co-trimethylene carbonate), polyphosphoesters, polyphosphoester urethanes, poly(amino acids), cyanoacrylates, poly(trimethylene carbonate), poly(iminocarbonate), copoly(ether-esters) (e.g. PEO/PLA), polyalkylene oxalates, polyphosphaZenes and biomolecules such as fibrin, fibrinogen, cellulose, starch, collagen, hyaluronic acid, etc., and mixtures thereof.

Matrixes containing the stressed serum need not contain cells. For instance, a matrix containing a sufficient amount of the stressed serum to increase recovery, viability and/or establishment of cells may be placed adjacent or otherwise in proximity the cells and/or organ and/or tissue to be treated. For instance, the stressed serum may be incorporated into a hydrogel placed beneath, over and/or in proximity to a cell containing graft and/or matrix. Matrixes other than hydrogels may also be used. For instance, the stressed serum may be incorporated into a bandage placed over and/or adjacent to implanted cells. It also possible for cells to seeded over a matrix containing a sufficient amount of the stressed serum. The matrix should be sufficiently porous and/or have sufficient internal spaces as to hold and elude a sufficient amount of the stressed serum to increase recovery and/or establishment of the implanted stem cells. The matrix may be bioabsorbable as eliminate the need for subsequent surgeries to retrieve the matrix. Additionally, absorption of the matrix by the body may facilitate release of a sufficient amount of the stressed serum over time. Appropriate bioabsorbable matrixes may be fabricated from poly(hydroxyvalerate), poly(L-lactic acid), polcaprolactone, poly(lactide-co-glycolide), poly(hydroxybutyrate), poly(hydroxybutyrate-co-valerate), polydioxanone, polyorthoesters, polyanhydrides, poly(glycolic acid), poly(D.L-lactic acid), poly(glycolic acid-co-trimethylene carbonate), polyphosphoesters, polyphosphoester urethanes, poly(amino acids), cyanoacrylates, poly(trimethylene carbonate), poly(iminocarbonate), copoly(ether-esters) (e.g. PEO/PLA), polyalkylene oxalates, polyphosphaZenes and biomolecules such as fibrin, fibrinogen, cellulose, starch, collagen, hyaluronic acid, etc., and mixtures thereof.

The stressed serum may also be combined with the cells by applying a physical structure containing a sufficient amount of the stressed serum, such as a gauze, bandage and/or dressing. For instance, a stressed serum derived from skin cells to be used with cells implanted into a wound may be incorporated into a wound dressing. The physical structure should be sufficiently porous and/or have sufficient internal spaces as to hold and elude a sufficient amount of the stressed serum to facilitate recovery and/or establishment of the implanted stem cells.

The stressed serum may also be incorporated into a solution sprayed onto implanted cells.

Some conditions may require a systemic application of stem cells. For instance, patients receiving chemotherapy may be administered intravenous injections of the stem cells to mitigate graft-versus-host-disease (GVHD). The stressed serum may be combined with cells for injection by including within an injectable solution containing the cells a sufficient amount of the stressed serum to increase recovery, viability and/or establishment of the cells. The amount of the stressed serum necessary to increase recovery, viability and/or establishment may not necessitate inclusion of the stressed serum within the injected solution. Simply making the stressed serum available during thawing of cryopreserved cells may be sufficient. Making the stressed serum available during thawing may be accomplished by including the stressed serum within the cryopreservation solution and/or thawing cryopreserved cells in a solution containing the stressed serum. Once thawed, the cells may be separated from the cryopreservation solution and combined with the injection solution.

The stressed serum may be obtained from stressing cultures comprising one or more cell types. When the stressed serum is to be combined with implanted cells, the cell culture may, but not need, contain the same type of cells to be implanted. For instance, when stem cells are utilized to produce the stressed serum, they may, but need not, be the same type of stem cells to be ultimately implanted.

The stressed serum may also be obtained from other cells. For instance, when cells are implanted to assist establishment of a graft, the stressed serum may be obtained from cells of the target organ. As organs are composed of different cell types acting in concert, collecting a stressed serum from a co-culture comprising various cells types of the organ into which the cells are implanted may better facilitate establishment, recovery and/or viability of the implanted cells.

Cells may be implanted into the skin to facilitate wound healing and/or acceptance of a skin graft. For instance, cells may be sprayed or otherwise laid over a wound prior to placement of graft tissue. Cells may also be implanted after placement of a tissue graft. Whether used alone or in combination with a graft, the goal is to have the cells facilitate healing and/or growth of healthy skin. Healthy skin comprises various cells, such as keratinocytes, fibroblasts, mesodermal cells, melanocytes, Merkel Cells, Langerhans cells, etc. Accordingly, collecting a stressed serum from a co-culture comprising keratinocytes, fibroblasts, mesodermal cells, melanocytes, Merkel Cells, Langerhans cells, T-cells and/or other skin cells in various combinations may provide a stressed serum better extending replenishment.

Similarly, collecting a stressed serum from a co-culture of hepatocytes, stellates, Kupffer cells and/or endothelial cells may better extend replenishment of cells implanted within the liver. Collecting a serum from a co-culture comprising all or a portion of the various cells within nervous system, likewise, may better extend replenishment of implanted cells and/or assist replenishment the nervous system. Likewise, a culture of cardiac cells may better facilitate establishment, recovery and/or viability of cells implanted within the heart and/or replenishment of the cardiac system. However, different growth rates and/or nutrients requirements may complicate efforts to co-culture different cells. A portion of the cells to be co-cultured, for instance, may only be able to survive in a growth medium that promotes the proliferation of other cells within the co-culture. With some cells proliferating and others only surviving, the cultures could be become dominated by the proliferating cells. That is, the resulting co-culture may comprise disproportionate amounts of the various cell types. Dominated by one type of cell over the other types, paracrine signaling may be altered, diminished and/or lost. Having altered and/or diminished paracrine signaling, such a culture would be unlikely to produce a stressed serum having all the proteins and/or other molecular factors necessary for extending replenishment and/or promoting recovery, viability and/or establishment of the implanted cells.

Dominance by one cell type, i.e. a disproportionate amount, within a co-culture may be avoided by seeding a first surface with a first culture of cells and allowing the culture to become established by growing the first culture to a monolayer of less than one-hundred percent confluence. Growing the first culture to a monolayer of less than one-hundred percent confluence may be facilitated by growing the first culture in the presence of a growth medium comprising nutrients and at least one growth factor. The first growth medium should promote the proliferation of the first cell culture. Growing the first culture to less than one-hundred percent confluence provides at least one cell free area on the first surface. A second culture of cells may then be seeded onto at least a portion of the cell free areas of the first surface. The second culture of cells may comprise cells different than the first culture. After being seeded onto at least one of the cell free surface areas of the first surface, the second culture and first culture can be grown to less than one-hundred percent confluence in the presence of a second growth medium. The second growth medium should promote the proliferation of the second culture and may comprise nutrients and at least one growth factor. Depending upon the cells within the first and second cultures, the first growth medium and second growth medium may be identical, include shared components and/or be completely different. Accordingly, the second growth medium may comprise the nutrients and at least one growth factor of the first growth medium. The procedure may be repeated with subsequent cell cultures and subsequent growth medium, which may allow for complex and/or diverse co-cultures. Following the general procedure of seeding subsequent cultures onto cell free areas of a surface may facilitate establishment of a co-culture by providing the subsequent cultures room and/or favorable conditions to become established. Favorable conditions may be provided by changing the growth medium and/or other conditions, such as temperature and/or pH, as to promote proliferation of the subsequently seeded cultures. Favorable conditions promoting the proliferation of subsequently seeded cultures should not be such as to cause previously seeded cultures to become senescent. The stressed serum collected from a co-culture, as well as a single culture, may be enhanced by maintaining the cells in a proliferative phase by growing the final co-culture to less than 100% confluence on the first surface after seeding the final culture to be added. Maintaining the cells in a proliferative phase prior to and/or during serum collection may influence the composition of the stressed serum.

Production of the stressed serum may be induced in a single cell culture or co-culture by replacing the final growth medium with a collection medium lacking all or a portion of the growth factors of the final growth medium, after the culture has been grown to less than one-hundred percent confluence. For instance, the final growth medium may be replaced with a collection medium lacking at least one of the growth factors of the final growth medium. The collection medium may comprise the nutrients of at least one of the final growth medium and/or other growth mediums used in establishing the culture. The culture may then be maintained for a period of time in the collection medium sufficient to allow the culture to produce from the collection medium a conditioned medium comprising the stressed serum. After which, at least a portion of the conditioned medium containing the stressed serum may be collected.

Facilitating growth of a monolayer may be accomplished by utilizing a surface enabling growth of the culture radially outwards as a single layer from each seeded cell and/or collection of cells. Seeding a surface comprises depositing cells of the culture to be grown onto the surface. Depending on the manner utilized to deposit cells onto the surface, the cells may be deposited as individuals or clusters. For instance, seeding the cells from a solution may cause individual cells to be deposited over the area of the surface seeded. In combination or the alternative, the surface may be seeded by depositing tissue samples and/or portions of a previously grown culture onto the surface. In such instances, clusters of cells may be deposited onto the area of the surface seeded. The surface should allow the culture to grow radially outwards from deposited cells. Of course, portions of the culture growing radially outwards from individually seeded cells and/or clusters may merge together. The cells may also coalesce together to form various three-dimensional configurations. Hepatocytes, for instance, may self-assemble into spheroids. Accordingly, a monolayer may comprise various three-dimensional aggregates, provided that such aggregates and other cells within the culture are predominately arranged as a single layer.

Radial growth from seeded cells and/or clusters providing a monolayer may be facilitated with a variety of surfaces. For instance, the bottom of Erlenmeyer flask and/or petri dish may provide a sufficient surface. The sides of T-flask may also provide a sufficient surface. When vessels such as a petri dishes and/or flasks are utilized, they should be sized to provide a sufficient volume of growth medium enabling growth of the final culture to be obtained.

The surface does not need to be the sides of a vessel. For instance, the surface may be one or more plates submerged in a growth medium.

The surface may also be provided by a gelling agent, such as agar. In such instances, the culture could be grown on the solidified gelling agent. The gelling agent may include all or a portion of the growth medium. When the gelling agent does not include the entire growth medium, the missing elements of the growth medium may be provided by submerging the solidified gelling agent in an appropriate solution.

As to facilitate growth and establishment, a first and/or subsequent culture may be provided with a growth medium comprising nutrients and growth factors. Growth factors are substances capable of stimulating healing, growth, cellular proliferation and/or cellular differentiation. Growth factors useful for facilitating growth and establishment of a culture of skin cells may include, but are not limited to, amino acids, such as L-Glutamine, hormones, such as hydrocortisone hemisuccinate, insulin and/or epinephrine, omega fatty acids, such as linoleic acid, vitamins, such as vitamin C, proteins, such as serum albumin, basic fibroblasts growth factor, acidic fibroblasts epidermal growth factor, transforming growth factor, insulin, bovine pituitary extract and/or ApoTransferin, and/or glycerophospholipids, such as lecithin. Other growth factors may facilitate the growth of the other cells to be cultured. Accordingly, the growth factors added may be chosen to promote the growth and/or proliferation of the cells to be cultured.

The nutrients in the growth medium provided to a culture need not be lavish or exceed the minimal nutrients required for survival and growth of the culture. As such, a basal medium and/or minimal essential medium can provide sufficient nutrients. The nutrients of the first, second and/or other subsequent growth medium, therefore, may be provided by a basal medium. Accordingly, the first, second and/or other subsequent growth medium provided to a culture may comprise growth factors combined with a basal medium and/or minimal essential medium. The growth medium provided to a first, second and/or other subsequent cultures may include a balancing agent to buffer the medium to a desired pH, such as, but not limited to, Earl's salts and/or 4-(2-hydroxyethyl)-1-piperazineethanesulfonic acid (HEPES).

Accordingly, when establishing a culture of cells, a first seeded culture may be provided with a growth medium comprising nutrients and at least one growth factors as to facilitate establishment of the culture after being seeded onto a surface. The growth medium provided to the first seeded culture may comprise a basal medium and/or minimal essential medium. The growth factors included within the growth medium provided to the first and/or subsequent seeded cultures may include, but are not limited to, amino acids, such as L-Glutamine, hormones, such as hydrocortisone hemisuccinate, insulin and/or epinephrine, omega fatty acids, such as linoleic acid, vitamins, such as vitamin C, proteins, such as serum albumin, basic fibroblasts growth factor, epidermal growth factor, transforming growth factor, insulin, bovine pituitary extract and/or ApoTransferin, and/or glycerophospholipids, such as lecithin.

Different growth rates and/or nutrients requirements may complicate efforts to co-culture different cells. Keratinocytes, for instance, may only be able to survive in a growth medium that promotes the proliferation of T cells. Consequently, attempting to co-culture T-cells with keratinocytes and/or other skin cells can easily result in a culture in which not all cell types are proliferating. With some cell types proliferating and others only surviving, the cultures could be become dominated by the proliferating cells. Being dominated by one type of cell over the other types, paracrine signaling may be altered, diminished and/or lost. Having altered and/or diminished intercellular signaling, such a culture would be unlikely to produce a stressed serum having all the proteins and/or other molecular factors necessary for extending replenishment and/or increasing recovery, viability and/or establishment of implanted cells.

For instance, traditional keratinocyte mediums, such as Dulbecco's Modified Eagle Media (DMEM), Keratinocyte Growth Medium (KGM, Lonza) and Roswell Park Memorial Institute medium (RMI medium), are effective for promoting the proliferation of keratinocytes. However, such mediums may not sufficiently promote the growth and survival and T-cells. The resulting co-culture, accordingly, may be dominated by keratinocytes. Attempting to co-culture keratinocytes and T-cells in a growth medium promoting the survival and growth of T-cells, such as Xvivo15 from Lonza, may not promote the proliferation of keratinocytes. Utilizing a medium not promoting the proliferation of keratinocytes, the resulting culture may be dominated by T-cells. Dominated by keratinocytes or T-cells, the natural paracrine signaling between the cells may be lost. Paracrine signaling between keratinocytes and T-cells may stimulate the production of molecular factors, such as cytokines like IL-17, useful for promoting homeostasis of the skin and defending against fungal and bacterial infections. A serum comprising such homeostasis and/or defensive molecular factors may be beneficial in promoting extending replenishment and/or increasing recovery, viability and/or establishment of cells implanted into the skin and/or other organs. Co-cultures dominated by keratinocytes or T-cells may not provide such beneficial serums due to paracrine signaling being altered, diminished and/or lost.

Dominance by one cell type within a co-culture may be avoided by seeding a surface with a first culture and growing the first culture to a monolayer of less than one-hundred percent confluence in the presence of a first growth medium comprising nutrients and at least one growth factor, as to provide at least one cell free area. All or a portion of the cell free areas of the first surface may then be seeded with a second culture. After seeding of the second culture, the first and second cultures may be grown to a monolayer of less than one-hundred percent confluence in the presence of a second growth medium comprising nutrients and at least one growth factor. The second growth medium should promote proliferation of the second culture. Accordingly, in the case of a co-culture of T-cells and keratinocytes, the first culture may comprise keratinocytes and the first growth medium may comprise a growth medium promoting the proliferation of the keratinocytes, such as DMEM, KGM or RMI medium. After the keratinocytes have been grown to a sufficient monolayer, the first surface may be washed to remove the first growth medium. All or a portion of the cell free areas on the first surface may then be seeded with a second culture comprising T-cells. The second and first culture may then be grown to a monolayer of less than one-hundred percent confluence in the presence of a second growth medium promoting the proliferation of T-cells, such as Xvivo15. As to maintain at least some level of proliferation of the first culture, the second medium may comprise at least one growth factor facilitating and/or promoting the proliferation of the first culture and/or intervening cultures. For instance, if the first culture comprises keratinocytes, the second growth medium may comprise at least one of bovine pituitary extract, L-glutamine, hydrocortisone hemisuccinate, transforming growth factor, insulin, epinephrine, and ApoTransferrin, or combinations thereof.

Different responses to growth factors may also complicate efforts to co-culture different types of cells. For instance, hepatocytes can spontaneously form spheroids when cultured. The formation of spheroids, however, is inhibited by fetal bovine serum. Obtaining a monolayer comprising hepatocytes spheroids, accordingly, requires limiting and/or precluding exposure to fetal bovine serum. Stellates, another cell of the liver, require the presence of fetal bovine serum to grow in culture. Providing a second culture comprising hepatocyte spheroids after a first culture of stellates may require replacing a first growth medium comprising fetal bovine serum with a second growth medium lacking fetal bovine serum. The second growth medium, therefore, may lack a nutrient and/or growth factor of the first growth medium.

Attempts to co-culture different types of cells may also be complicated by different growth rates in response to the growth medium. A growth medium including basic fibroblast growth factor (bFGF) stimulates proliferation of fibroblasts more than keratinocytes. Conversely, acidic FGF stimulates proliferation of keratinocytes more than fibroblasts. As such, an acidic growth medium may cause a co-culture of fibroblast and keratinocytes to be dominated by keratinocytes, whereas a basic growth medium may cause the co-culture to be dominated by fibroblast. Being dominated by fibroblast over keratinocytes, or vice versa, paracrine signaling may be altered, diminished and/or lost. Having altered and/or diminished paracrine signaling, such a culture would be unlikely to produce a serum having all the proteins and/or other molecular factors necessary for extending replenishment and/or increasing recovery, viability and/or establishment of implanted cells.

Dominance by one cell type over another due to different responses to growth factors may be lessened and/or avoided by removing growth factors favoring proliferation of previous seeded cultures prior to growing later seeded cultures. In combination or the alternative, the growth factors favoring the later seeded culture may be added to the growth medium. Accordingly, the first growth medium may comprise a growth factor promoting the proliferation of a first seeded culture. In some instances, the first growth medium may comprise growth factors promoting the proliferation of the first culture more than subsequent cultures. For instance, if the first culture comprises fibroblast cells, the first growth medium may comprise bFGF. After the first seeded culture has been grown to a monolayer of less than one-hundred percent confluence, as to provide at least one cell free area, the first surface may be washed to remove the first growth medium. A second cell culture may then be seeded onto all or a portion of the cell free area. The first and second cultures may then be grown to a monolayer of less than one-hundred percent confluence in the presence of a second growth medium. The second growth medium may comprise a growth factor promoting the proliferation of cells within the second culture more than cells within the first culture. For instance, if the second culture comprise keratinocytes, the second growth medium may comprise aFGF. As to maintain at least some level of proliferation of the first culture, the second medium may comprise at least one growth factor facilitating and/or promoting the proliferation of the first culture and/or intervening cultures.

Attempts to co-culture different types of cells may also be complicated by different growth rates of the cells, regardless of the growth medium. Some cells of the intended co-culture may be slow to proliferate with respect to other cells of the co-culture across many different growth mediums. Growing quicker than other cells within the co-culture, the faster proliferating cells may crowd out the other cells within the co-culture. Consequently, attempting to co-culture faster proliferating cells with slower proliferating cells may result in a co-culture dominated by the faster proliferating cells over the slower proliferating cells.

Dominance by one cell type over another due to different growth rates may be lessened and/or avoided by allowing slower growing cells to create a sufficient monolayer on a surface prior to seeding faster growing cells onto the surface. For instance, the first seeded culture may comprise cells with slower doubling times than other cells to be cultured. After the first seeded culture has been sufficiently established on the surface, cultures comprising more proliferative cells can be seeded onto at least one cell free area on the surface to provide a co-culture. Once the more proliferative cells have been established, subsequent cultures can be seeded and established until a co-culture having the desired cellular composition is obtained on the surface. Achieving the desired cellular composition of the final co-culture may require seeding successive cultures onto the surface at varying confluences. For example, if the final co-culture is to comprise a co-culture of a first cell type, a second cell type with faster growth rate than the first cell type, and a third cell type with a faster growth rate than the first and second cell types, then it may be appropriate to first seed a culture of the slowest growing cells onto the surface and grow the first seeded culture to approximately thirty-three percent confluence. A culture of cells having the intermediate growth rate may then be seeded onto at least one cell free area on the first surface. The co-culture of the first and second cell types may then be grown to approximately sixty-six percent confluence. Then a culture of the fastest growing cell types may be seeded onto at least one cell free area on the first surface and the co-culture of the three cells grown to less than one-hundred percent confluence.

The above examples are based upon situations in which subsequent seeded cultures comprise cells growing sufficiently faster than previously seeded cultures such that the growth of previously seeded cultures can be treated as halted, and that a final co-culture having equal amounts of each culture is desired. One or both these assumptions may not be true for every co-culture to be produced. Accordingly, it may be advantageous to grow the first seeded culture and/or subsequent co-cultures to other confluences as to account for growth rates of previously seeded cultures with subsequently seeded cultures and/or the desired cellular composition of the final co-culture.

The composition of a stressed serum generated by a culture of cells may also be altered by cells becoming senescent. Cells are more productive when they are actively proliferating. Additionally, cells in the proliferative phase produce different molecular factors then senescent cells. Having altered and/or diminished production of molecular factors, a culture comprising senescent cells may also be unlikely to produce a stressed serum having all the proteins and/or other molecular factors necessary extending replenishment and/or increasing recovery, viability and/or establishment of implanted cells. Therefore, it may be desirable to maintain cultures from which cells are to be harvested for culturing in a proliferative phase. Likewise, it may be desirable to maintain cells within seeded cultures in a proliferative phase.

Maintaining cells within a culture in a proliferative phase may be accomplished by preventing the culture form achieving one-hundred percent confluence. Accordingly, as to maintain the cells of the first seeded culture in a proliferative phase, the first seeded culture should be grown to a monolayer of less than one-hundred percent confluence, as to provide at least one cell free area on the surface. After the first culture has been established, a second culture may be seeded onto all or a portion of the cell free areas. The first and/or second culture may then be grown to less than one hundred percent confluence.

The cells of the second or other subsequent cultures seeded onto the surface having a monolayer of preceding cultures may be provided in suspension. The suspension may be acquired from one or more separately grown cultures. Such separate cultures may be grown on a surface and/or in suspension in the presence of a growth medium including growth factors and nutrients. The nutrients provided in the growth medium of the cultures from which cells to be seeded are harvested do not need to be lavish or exceed the minimal nutrients required for survival and growth of the culture. As such, a basal medium and/or minimal essential medium can provide sufficient nutrients. Accordingly, the growth medium provided to cultures from which cells are harvested may comprise growth factors combined with a basal medium and/or minimal essential medium. The growth medium provided to subsequent cultures may include a balancing agent to buffer the medium to a desired pH, such as, but not limited to, Earl's salts and/or HEPES.

Growth of subsequent cultures with the first culture after seeding may be facilitated and/or enhanced by maintaining the cells of subsequent cultures in a proliferative phase. Cells of subsequent cultures may be maintained in a proliferative phase by growing the cells on a second surface to less than one-hundred percent confluence prior to seeding onto the first surface of the co-culture. For instance, growing the cells of subsequent cultures on a second surface to approximately 80 to 90 percent confluence before seeding onto the surface of the co-culture may maintain the cells of the subsequent culture in a proliferative phase prior to seeding.

As to provide subsequent cultures a sufficient surface to become established, the first culture and/or preceding co-cultures are grown to monolayers of less than one-hundred percent confluence. For instance, the first culture and/or proceeding co-cultures may be grown to a monolayer of approximately eighty to ninety percent confluence, before seeding the second and/or subsequent cultures onto the at least one of the cell free areas of the first surface. Though previous cultures are grown to less than one-hundred percent confluence, it may be necessary to provide areas on the surface free of preceding cultures. Providing such surfaces within a monolayer of established cultures may be accomplished by growing preceding cultures to a monolayer of less than one-hundred percent confluence and then increasing the cell free surface area by creating voids in the monolayer of the first culture and/or preceding co-cultures by removing an appropriate amount of the monolayer. The amount removed will be dependent on the growth rate of the cells together and/or the final cellular composition of the co-culture desired. For example, if a co-culture comprising approximately equally amounts of two cell types is desired, and the second seeded cell type is sufficiently aggressively as to fill voids without allowing the first cells to significantly enter the voids, then approximately 50% of the monolayer of the first culture may be removed.

Voids may be created by removing portions of the monolayer of preceding cultures from the surface. For instance, voids may be created by scraping or otherwise mechanically detaching portions of the monolayer of the first culture and/or preceding co-cultures from the surface. It is also possible to remove portions of an established monolayer by first treating the monolayer with a detachment solution for a sufficient time to cause cells in the monolayer to begin to ball. Once the cells of the monolayer begin to ball, the detachment solution can be withdrawn and squirted back onto the monolayer to produce voids in the monolayer.

The serum collected from culture of cells may be enhanced by maintaining the cells in a proliferative phase by growing the final culture to less than one-hundred percent confluence on the first surface after seeding the final culture to be added. Maintaining the cells in a proliferative phase prior to and/or during serum collection may influence the composition of the stressed serum. The composition of the stressed serum may be adjusted by allowing at least a portion of the cells to senesce. Accordingly, the composition and/or ratio of senesced versus proliferative cells within the culture may influence the composition and/or quality of the stressed serum collected.

Cells within the culture may be stressed by selectively depriving the cells of at least one growth factor, nutrient and/or metabolic component. For example, cells within the culture may be stressed by depriving the cells of one or more growth factors while maintaining nutrient levels. Cells within the culture may by stressed after the culture has grown to approximately 80 to 95% confluence or at other times. Accordingly, production of a stressed serum may be induced in a culture by replacing the first and/or second growth medium with a collection medium lacking all or a portion of the growth factors of the final growth medium, after the final culture has been grown to less than one-hundred percent confluence. For instance, in a co-culture, the second growth medium may be replaced with a collection medium lacking at least one of the growth factors of the second growth medium. The collection medium may comprise the nutrients of at least one of the first and second growth mediums. The culture may then be maintained for a period of time in the collection medium sufficient to allow the culture to produce from the collection medium a conditioned medium comprising the stressed serum. After which, at least a portion of the conditioned medium containing the stressed serum may be collected.

As to facilitate further production of the stressed serum, the withdrawn portion of the conditioned medium may be replaced with fresh collection medium. Production of the stressed serum may also be enhanced by allowing the culture to recover from the induced stress. Such a recovery phase may be provided by replacing the conditioned medium with a recovery medium containing the removed growth factors and culturing the culture in the recover medium for a recovery period of time. Preventing the culture from obtaining one-hundred percent confluence during recovery may improve and/or maintain the quality of the stressed serum produced. The recovery medium may comprise the growth factors and/or nutrients of at least one of the first, second and other growth medium utilized in establishing the culture. Depending on the cells included within the culture, a recovery period of approximately 24 to 72 hours may be sufficient.

The collected stressed serum may be combined with cells prior to and/or after implantation. When the cells are to be cryopreserved, the stressed serum may combined with the cells prior to and/or after cryopreservation. As such, the stressed serum may be incorporated into a cryopreservation solution. In combination or the alternative, the stressed serum may be combined with the cells when the stem cells are thawed. Accordingly, the cells may be thawed in solution containing the stressed serum. The stressed serum may also be combined with the stem cells after thawing.

As to better match the individual to be treated, the culture of cells providing the stressed serum may be harvested from the patient to be treated and/or a related individual.

The stressed serum may also by synthetic generated by combining a sufficient amount of at a least one an angio-modifying formulation, a morphogenesis formulation, an extracellular matrix formulation, an extracellular matrix modification formulation, an immune promoting formulation and a cytoskeleton formulation with a suitable solvent, such as water. A sufficient amount refers to an amount of stressed serum extending replenishment by increasing at least one establishment, recovery, viability, growth, migration and/or differential of cells. When combined with cells, a sufficient amount refers to amount sufficient to increase recovery, viability and/or establishment of the cells. The angio-modifying formulation, morphogenesis formulation, extracellular matrix formulation, extracellular matrix modification formulation, immune promoting formulation and/or cytoskeleton formulation may comprise proteins having catalytic, structural, regulatory and/or messaging functions. Proteins with a catalytic function may catalyze reactions. For instance, catalytic proteins may catalyze the degradation of the extracellular matrix allowing for increased establishment. Structural proteins within one or more of the formulations may become incorporated into the structure and arrangement of the tissue and/or organ to be treated and/or the implanted cells. For instance, structural proteins may become part of the cytoskeleton, extracellular matrix and/or junctions between cells. Structural proteins may also anchor and/or facilitate migration of implanted cells and/or newly formed cells. Regulatory proteins within the formulations may control the action of proteins within the organ and/or tissue to be treated and/or the implanted cells. For instance, a regulatory protein may activate or inhibit a catalytic protein. Proteins providing messaging functions may be responsible for activating processes within the implanted cells and/or tissue and/or organ to be treated. A messaging protein within the formulations, for instance, may bind to receptors within the receiving tissue and/or implanted cells to elicit and/or inhibit various cellular process facilitating replenishment and/or recovery, viability and/or establishment of implanted cells. For instance, a messaging protein may act attract immune cells that protect implanted cells.

As the varied actions of proteins that may be present within the formulations of the synthetically derived stressed serum demonstrate, extending paracrine signaling and/or increasing recovery, viability and/or establishment of implanted cells may be accomplished by augmenting paracrine signaling naturally present. The stressed serum may augment paracrine signaling by amplifying natural paracrine signaling. A stressed serum amplifying natural paracrine would comprise at least one formulation having at least one messenger protein of natural paracrine signaling. Generally, cells respond proportionally to the amount of the messenger proteins received. Accordingly, if more messenger proteins were present, then the cell would produce a bigger response. A stressed serum comprising a messenger protein of natural paracrine signaling would, consequently, augment paracrine signaling by increasing one aspect of natural replenishment. In addition, or the alternative, to amplifying one aspect of natural paracrine signaling, the stressed serum may augment paracrine signaling to extend replenishment and/or increase recovery, viability and/or establishment of implanted cells by providing catalytic activities, regulation, messages and/or structural changes not generally induced during replenishment. It is also possible to augment paracrine signaling by providing proteins naturally produced by paracrine signaling. A stressed serum may augment paracrine to extend replenishment of organs and/or tissue and/or increase recovery, viability and/or establishment of implanted cells through any combination of these mechanisms.

During paracrine signaling, molecules communicating massages about the environment and/or health of an organ and/or tissue move between cells and layers. When the signaling molecules reach their target cells and/or layers, an appropriate response is induced in the receiving cell. The molecules may travel between cells and/or layers by diffusion, i.e. movement through a fluid. Thus, to have proper paracrine signaling in the skin, for instance, the skin needs to remain hydrated, as to provide a fluid through which the signaling molecules can move. If the skin were to become dry, the loss of fluid would limit the diffusion of signaling molecules. The reduced diffusion of signaling molecules in dry skin may delay, diminish and/or prevent paracrine signaling, thereby adversely influencing replenishment and/or viability, recovery and/or establishment of implanted cells. A capillary bed beneath the skin supplies the skin with nutrients and moisture, keeping the skin hydrated and nourished. Modifying the capillary bed to increase its ability to supply the skin with moisture and nutrients may, therefore, augment paracrine signaling to extend replenishment and/or increase viability, recovery and/or establishment of implanted cells. Accordingly, augmenting paracrine signaling to extend replenishment and/or increase viability, recovery and/or establishment of implanted cells may be accomplished by combining the stem cells with a stressed serum comprising a sufficient amount of angio-modifying formulation comprising agrin and calpastatin.

The ability of calpastatin in combination with agrin to augment paracrine signaling to by modifying the capillary bed is unexpected. Calpastatin has been shown to impair angiogenesis. Impairing angiogenesis, calpastatin would be expect to prevent modification of the capillary bed, resulting in impaired recovery and/or establishment of implanted stem cells. Stressed serum comprising calpastatin and agrin, however, extend replenishment of tissue, providing a better environment for implanted cells to recover, remain viable and/or establish themselves.

In addition to agrin and calpastatin, the angio-modifying formulation may comprise at least one of protein Jagged-1, Isoform 2 of growth arrest-specific protein 6, vascular endothelial growth factor C, 72 kDa type IV collagenase, desmoglein-2.

In addition to facilitating the movement of signaling molecules, paracrine signaling extending replenishment and/or increasing viability, recovery and/or establishment of implanted cells should induce cell mobility and differentiation. As organs and/or tissues replenish themselves with new cells, the newly formed cells move from their point of origin to replace lost cells. As the newly formed cells move, they differentiate, i.e. undergo morphological changes, to develop the morphology characteristic of the cells they are replacing. Accordingly, replenishment of an organ and/or tissue may be extend and/or viability, establishment and/or recovery of implanted cells may be increased by administering to the tissue and/or organ a sufficient amount of stressed serum. Likewise, providing implanted stem cells with morphological proteins promoting differentiation and cell mobility may augment paracrine signaling to extend replenishment and/or improve recovery, viability and/or establishment of implanted cells. Fibronectin, a protein of the extracellular matrix, facilitates cell mobility by providing footholds for moving cells. Tenascin, however, inhibits cellular adhesion to fibronectin. Accordingly, it would be expected that tenascin would inhibit cell mobility by causing the implanted stem cells to fall off fibronectin. Stressed serum comprising tenascin isoform 4 and Fibronectin isoform 3, however, improve cellular replacement. Augmenting paracrine signaling to extend replenishment and/or increase viability, establishment and/or recover of implanted cells, therefore, may be accomplished with a stressed serum comprising a sufficient amount of a morphogenesis formulation comprising tenascin isoform 4 and fibronectin isoform 3. Furthermore, combining stem cells with a stressed serum comprising a sufficient amount of a morphogenesis formulation comprising tenascin isoform 4 and fibronectin isoform 3 extends replenishment of tissue, providing a better environment for implanted cells to recover, remain viable and/or establish themselves.

In addition to tenascin isoform 4 and fibronectin isoform 3, the morphogenesis formulation may comprise at least one of transforming growth factor beta induced protein ig-h3, plasminogen activator inhibitor 1, amyloid beta A4 protein, glucose-6-phosphate isomerase, long isoform of serine protease inhibitor Kazal-type 5, cadherin-3, pappalysin-1, insulin-like growth factor-binding protein 7, kallikrein-10, protocadherin fat 1, syntenin-1, proliferation-associated protein 2G4 protein CYR61, keratinocyte proline-rich protein, brain-specific serein protease 4, cadherin 13 isoform 4, integrin alpha-2, integrin beta-1, and neural cell adhesion molecule L1 isoform 2.

In addition to facilitating morphogenesis of cells and/or movement of signaling molecules, extending replenishment and/or increasing viability, establishment and/or recovery of implanted cells may be accomplished by augmenting the integrity of the extracellular matrix, so that replenished and/or implanted cells are not lost to rapidly. The extracellular matrix is a scaffolding holding tissue and organs together. If this scaffolding were to weaken, implanted and/or replenished cells would quickly be lost. One of the proteins holding cells to the extracellular matrix is laminin. Augmenting paracrine signaling to extend replenishment and/or increase viability, recovery and/or establishment of implanted cells, accordingly, may also be accomplished by maintaining the integrity of the extracellular matrix. As such, a stressed serum having a sufficient amount of an extracellular matrix formulation comprising at least one laminin alpha 3, laminin beta 3, laminin beta 2 and laminin 332 may extend replenishment of organs and/or tissue and/or increase viability, recovery and/or establishment of implanted cells. Furthermore, combining cells with a stressed serum comprising a sufficient amount an extracellular matrix formulation comprising at least one laminin alpha 3, laminin beta 3, laminin beta 2 and laminin 332 to the skin may extend replenishment and/or increase recovery, viability and/or establishment of implanted stem cells.

In addition to the afore mentioned laminins, the extracellular matrix formulation may comprise at least one of collagen alpha-1(I), collagen alpha-1(III), collagen alpha-1(VI), collagen alpha-2(I), collagen alpha-2(VI), laminin subunit alpha 4, laminin subunit beta 1, laminin subunit gamma 1, and laminin 411.

While the integrity of the extracellular matrix is important, so too is the integrity of newly formed cells. A key component of cellular strength is the cytoskeleton. The cytoskeleton is a series of interconnected proteins assisting cells in maintaining their integrity and shape. Additionally, proteins within the cytoskeleton may facilitate cell mobility. Accordingly, augmenting paracrine signaling to extend replenishment and/or increase viability, recovery and/or establishment of implanted cells may be accomplished with a stressed serum comprising a sufficient amount of a cytoskeleton formulation. Furthermore, combining cells with a stressed serum comprising a sufficient amount of a cytoskeleton formulation to the skin may increase recovery, viability and/or establishment. The cytoskeleton formulation may comprise at least one of translationally-controlled tumor protein, filamin A, alpha-actin-1, microtubule associated protein 4, moesin, vinculin in a peptide, involucrin, gelsolin isoform 2, PDZ and LIM domain protein 1, caldesmon isoform 5, LIM domain and actin-binding protein 1, myosin regulatory light chain 12B, small proline rich protein 3, smooth muscle isoform of myosin light polypeptide 6, jupiter microtubule associated homolog 1, small proline rich protein 2A, myotrophin.

Although preserving and/or increasing the integrity of the tissue, organs and/or cells may augment paracrine signaling to extend replenishment and/or increase viability, recovery and/or establishment of implanted cells, shedding of dead cells, removal of toxins and removal of microorganisms may also be beneficial. Generally, such housekeeping activities result from an inflammation response induced by the complement system of the immune system. Inducing inflammation and/or other immune responses seems counterintuitive. After all, inflammation is generally characterized by redness and burning, itching and/or painful sensations. Given these negative effects of inflammation and other immune responses, one would suspect that including within the stressed serum an immune promoting formulation would lead to a decrease in replenishment and/or decreased recovery, viability and/or establishment of implanted cells. Despite these negative expectations, stressed serums comprising serapin B7, complement component C1s and complement component C3 improve replenishment of the tissue, providing a better environment for recovery, viability and/or establishment of implanted cells. Perhaps, the extended replenishment results from an increased removal of old and/or diminished cells. Augmenting paracrine signaling to extend replenishment and/or increase viability, establishment and/or recovery of implanted cells, therefore, may be accomplished with a stressed serum comprising a sufficient amount of an immune promoting formulation comprising serapin B7, complement component C1s and complement component C3 improve replenishment of the skin. Furthermore, combining stem cells with a stressed serum comprising a sufficient amount of an immune promoting formulation comprising serapin B7, complement component C1s and complement component C3 to the may increase recovery, viability and/or establishment.

In addition to serapin B7, complement component C1s and complement component C3, the immune promoting formulation may comprise at least one of neutrophil chemotactic agent, such as, but not limited to, interleukin-6, growth regulated alpha protein, protein S100-A8, interleukin-8, and/or C—X—C motif chemokine 5, and/or at least one of elafin, matrix metalloproteinase-9, stromelysin-2, HLA class I histocompatibility antigen, Cw-6 alpha chain, quinone oxidoreductase PIG3, superoxide dismutase, metallothionein-2, alpha-1 antichymotrypsin, interleukin-1 receptor-like 1.

Just providing the organ and/or tissue to be treated with extracellular matrix proteins, immune promoting proteins, angio-modifying proteins and/or cytoskeleton proteins may not be sufficient to augment paracrine signaling to extend replenishment and/or increase recovery, viability and/or establishment of implanted cells. The extracellular matrix proteins provided by the stressed serum, synthesized by the organ and/or synthesized by the implanted stem cells may not be readily incorporated into the existing extracellular matrix. It is also possible that newly formed cells and/or implanted cells may need assistance traversing the extracellular matrix. Likewise, neutrophils and/or other immune cells may need help infiltrating the tissue to clear a path for the implanted cells. Additionally, the extracellular matrix may inhibit modification of the capillary bed and/or delivery of nutrients and/or moisture to the treated organ and/or tissue. Such barriers may be overcome by including within the stressed serum extracellular matrix modifying proteins. The catalytic activity provided, directly and/or indirectly, by these proteins may facilitate incorporating new proteins of the extracellular matrix. In combination or the alternative, these proteins may facilitate passage of nutrients, moisture and/or cells. Augmenting paracrine signaling to extend replenishment and/or increase viability, establishment and/or recovery of implanted cells, accordingly, may also be accomplished by a stressed serum having a sufficient amount of an extracellular matrix modification formulation comprising at least one interstitial collagenase and stomelysin-1. Furthermore, combining cells with a stressed serum having a sufficient amount of an extracellular matrix modification formulation comprising at least one interstitial collagenase and stomelysin-1 may increase recovery, viability and/or establishment.

In addition to interstitial collagenase and stomelysin-1, the extracellular matrix modification formulation may comprise at least one of cathepsin L2, latent transforming growth factor beta-binding protein 2, aminopeptidase N, decorin, urokinase-type plasminogen activator, lumican, cystatin-M, lysyl oxidase homolog 2, cystatin-C, protein-lysine 6-oxidase, tissue factor pathway inhibitor 2, procollagen-lysine, 2-oxoglutarate 5-dioxgenase isoform 2.

### DETAILED DESCRIPTION

A method for obtaining a stressed serum suitable for administration to organs and/or tissue with or without cells to be implanted will be described more fully with reference to specific examples. The serum, however, may be obtained in different manners, and thus should not be construed as limited to the specific examples provided. Accordingly, the serum may be obtained by a different ordering and/or sequence of the various steps and/or procedures detailed in the provided examples. For example, two or more steps may be performed concurrently or with partial concurrence. Also, some steps that are performed as discrete steps in the following examples may be combined, and steps being performed as a combined step may be separated into discrete steps, the sequence of certain steps may be reversed or otherwise varied, and the nature or number of discrete steps may be altered or varied. Accordingly, the provided examples are not intended to exclude any of such means of obtaining a stressed serum suitable for in combination with implanted stem cells.

Likewise, different reagents, techniques, materials and/or equipment other than those specifically mentioned may be utilized to provide the stressed serum.

A stressed serum may be produced by stressing a culture including proliferative cells. The culture of cells may be obtained by first establishing a monolayer of a first cell culture on a surface. After a monolayer of a first culture is established, a second culture may then be seeded onto cell free areas within the monolayer and established. Additional cultures may then be seeded and established until a monolayer the having the desired cellular composition is obtained. The monolayer of the final culture is then stressed to obtain a serum by conditioning a collection medium. The obtained stressed serum may then be administered to an organ and/or tissue to be treated and/or combined with cells to be implanted before cryopreservation, during thawing, prior to implantation and/or after implantation. When combined prior to preservation, the stressed serum may be incorporated into the cryopreservation solution. When combined during thawing, cryopreserved cells may be thawed in a solution containing the stressed serum. When combined prior to implantation, the stressed serum may be incorporated into a solution, matrix and/or graft containing the cells. When combined after implantation, the stressed serum may be incorporated into a matrix placed over, under and/or adjacent the implanted cells and/or a solution sprayed onto the implanted cells.

A stressed serum may be obtained from a proliferative monolayer comprising a culture of one or more cell types, such as, but not limited to, a proliferative monolayer of keratinocytes and fibroblasts. The co-cultured monolayer may be established by first partially submerging a vial of frozen keratinocytes (obtained from LifeLine Cell Technologies) in a 37° C. water bath, without submerging the top of the vial. The vial is allowed to thaw in the water bath until a small piece of ice remains. The vial is then removed and sprayed with an ethanol solution. In a hood, keratinocytes are seeded from the vial at 2,500 to 5,000 cells per cm² onto a culture treated surface. The surface should be provided with an appropriate volume of a suitable growth medium, such as a medium including Basal DermaLife Media (LifeLine Cell Technologies) and growth factors comprising bovine pituitary extract, L-glutamine, hydrocortisone hemisuccinate, transforming growth factor, insulin, epinephrine and/or ApoTransferrin. The seeded surface is then placed in an incubator and grown at 37° C. in the presence of humidified air comprising 5% CO₂. As to remove any residue DMSO and/or other solvents that may be present in the cryogenic solution, the growth medium may be changed every 24 to 48 hours following initiation of the monoculture. After which time, the growth medium may be changed every 48 to 72 hours.

Other means of obtaining the initial keratinocytes may also be employed. For instance, keratinocytes may be isolated from neonatal foreskin retrieved from circumcision using the techniques detailed in U.S. application Ser. No. 14/597,796, filed Jan. 15, 2015, the teachings of which are hereby incorporated by reference in their entirety.

The keratinocytes are allowed to grow in the growth medium until 80-90% confluence is achieved. Voids are then created within the established monolayer by removing the growth medium and washing twice with an appropriate volume of a buffer solution, such as phosphate buffer solution without calcium or magnesium. After washing with buffer solution, a sufficient volume of an enzymatic cell detachment solution to promote detachment of the cells from the surface is added. The cell detachment solution may comprise proteolytic and/or collagenolytic enzymes. For instance, detachment of the cells may be promoted by adding 1 ml of Accutase Solution, available for Innovative Cell Technologies, Inc, per 25 cm² of growth area. The enzymatically treated monolayer may then be incubated at 37° C. until the keratinocytes start balling. The surface is then tilted to collect the enzyme solution with a pipette. The collected solution is sprayed at focused points onto the monolayer to create voids in about 50% of the monolayer. The enzyme solution and detached keratinocytes are then removed. The remaining monolayer is provided with a sufficient volume of the growth medium and returned to the incubator. For example, an amount of medium providing 10 ml of medium per 55 cm² of growth area may be sufficient.

Simultaneously, a monolayer of fibroblasts is cultured on a second surface by submerging a vial of frozen fibroblasts (obtained from LifeLine Cell Technologies) in a 37° C. water bath, without submerging the top of the vial. The vial is allowed to thaw in the water bath until a small piece of ice remains. The vial is then removed and sprayed with an ethanol solution. In a hood, fibroblasts are seeded from the vial at 2,500 to 5,000 cells per cm² on to a culture treated surface. The surface should be provided with an appropriate volume of a suitable growth medium, such as a medium including Basal DermaLife Media (LifeLine Cell Technologies) and growth factors comprising L-glutamine, hydrocortisone hemisuccinate, lineolic acid, licithin, human serum albumin, basic fibroblasts growth factor, epidermal growth factor, transforming growth factor, insulin and/or vitamin C. The seeded surface is then placed in an incubator and grown at 37° C. in the presence of humidified air comprising 5% CO₂. As to remove any residue DMSO and/or other solvents that may be present in the cryogenic solution, the growth medium may be changed every 24 to 48 hours following initiation of the monoculture. After which time, the growth medium may be changed every 48 to 72 hours.

Other means of obtaining the initial fibroblasts may also be employed. For instance, fibroblasts may be isolated from neonatal foreskin retrieved from circumcision using the techniques detailed in co-pending U.S. application Ser. No. 14/597,796.

When the fibroblast monolayer reaches approximately 80 to 90% confluence, the surface is transferred to a hood and the growth medium removed. The fibroblasts monolayer is then washed with a buffer solution, such as phosphate buffer solution without calcium or magnesium. A sufficient volume of a cell detachment solution to promote detachment of the fibroblasts from the second surface is then added. For instance, detachment of the fibroblasts may be promoted by adding 1 ml of Accutase Cell Detachment Solution, manufactured by Innovative Cell Technologies, Inc, per 25 cm² of growth area. The fibroblast cells are then incubated in the cell detachment solution at 37° C. until all the cells have detached. A homogenous suspension of cells is then obtained by mixing and the fibroblasts suspension is seeded onto cell free areas within the keratinocyte monolayer. The seeded culture is then returned to the incubator. The co-culture is then grown in the keratinocyte growth medium until 80 to 95% confluence is achieved.

A monolayer of co-cultured cells may also be achieved by culturing keratinocytes in the keratinocyte growth medium until approximately 50% confluence is achieved. Cell free areas on the surface may then be seeded with the cultured fibroblasts suspension. For example, a co-culture in a T175 cm² flask would be overlayed with 1.5 ml of fibroblast suspension generated from a confluent T75 cm² flask of fibroblasts dissociated using 3 ml of Accutase. The co-culture may then be grown in the incubator until approximately 80 to 95% confluence is achieved.

The co-culture of cells may be stressed to provide a therapeutic serum suitable for use in a cosmetic preparation. Stressing the co-culture may be achieved by selectively removing nutrients, growth factors and/or other favorable conditions. The stress need not be severe. Accordingly, sufficient stress may be induced by removing all or a portion of the growth factors while maintaining nutrient levels. Growth factors may be removed by extracting the keratinocyte growth medium from the surface and rinsing the co-culture monolayer twice with a sufficient volume of a buffer solution, such as phosphate buffer solution lacking calcium and magnesium. As to ensure all growth factors are removed, the co-culture may be incubated for a period of time in a collection medium that is added to the surface and then discarded prior to serum collection. For instance, growth factors may be removed prior to serum collection by adding approximately 5.0 ml of a collection medium per 55 cm² of growth area and incubating for approximately six hours.

The collection medium may comprise a minimum essential medium with Earl's salt and have the nutrients of the keratinocyte growth medium.

After removal of the growth factors, a sufficient volume of fresh collection medium is added, and the surface returned to the incubator for a sufficient period of time to produce a conditioned medium form the collection medium. For example, incubating the co-culture in approximately 10.0 ml of fresh collection medium per 55 cm² of growth area for approximately 48 hours may be sufficient to produce a conditioned medium from the collection medium. After incubating for a sufficient period of time, approximately 50% of the collection medium is removed and replaced with an approximately equal amount of fresh collection medium. The co-culture is then incubated for approximately 48 hours to produce more conditioned medium. After which time, all of the conditioned medium is removed.

The co-culture is then allowed to recover by removing the stress and incubating for a period of time. For instance, incubating in the presence of approximately 10.0 ml per 55 cm² of growth area of the keratinocyte growth medium for approximately 24 to 72 hours may provide sufficient recovery. During recovery, the co-culture may be refreshed by seeding fresh cells of one or more of the cultures onto the monolayer.

After recovering, serum collection is repeated.

The process of the serum collection and recovery may be repeated until the co-cultures no longer produce serum of the desired quality. For instance, three passes may be utilized. The quality of serum may begin to degrade when one or more of the cultures used to initially establish the co-culture reach 80% of their life expectancy as defined by the maximum number of population doublings.

The conditioned medium collected may be filtered using a suitable filter, such as a 0.45 μm Millipore filter. The serum collected from filtering the conditioned medium may be tested for sterility, virology and/or stability factors. Depending on the intended use of the serum, such testing may not be necessary.

The stressed serum may also be synthetically generated by combining formulations of proteins with a suitable solvent, such as water. The proteins of each formulation may be obtained from various suppliers and/or harvested from various organisms. The proteins may also be isolated from serums created by cell cultures.

The stressed serum may comprise a sufficient amount of an angio-modifying formulation comprising agrin isoform 6 and calpastatin isoform 6. The amount of agrin peptides may be less than the amount of calpastatin peptides within the formulation, with a preferred ratio of 8:13.

In addition to agrin isoform 6 and calpastatin isoform 6, the angio-modifying formulation may further comprise at least one of:
Protein Jagged-1, with the amount of protein-jagged-1 peptides being less than the amount of calpastatin peptides and less than the amount of agrin peptides, a preferred ratio of 5:13 with respect to calpastatin peptides;
Isoform 2 of Growth arrest-specific protein 6, with the amount of growth arrest-specific protein 6 peptides being less than the amount of calpastatin peptides and less than the amount of agrin peptides, a preferred ratio of 7:13 with respect to calpastatin peptides;
Vascular endothelial growth factor C, with the amount of vascular endothelial growth factor peptides being less than the amount of calpastatin peptides and more than the amount of agrin peptides, a preferred ratio of 10:13 with respect to calpastatin peptides;
72 kDa type IV collagenase, with the amount of collagenase peptides being greater than the amount of calpastatin peptides and more than the amount of agrin peptides, a preferred ratio of 22:13 with respect to calpastatin peptides; and
Desmoglein-2, with the amount of desmoglein-2 peptides being less than the amount of calpastatin peptides and less than the amount of agrin peptides, a preferred ratio of 3:13 with respect to calpastatin peptides.

The stressed serum may comprise a sufficient amount of morphogenesis formulation comprising tenascin isoform 4 and fibronectin isoform 3. The amount of tenascin peptides may be less than the amount of fibronectin peptides, with a preferred ratio of 8:41.

In addition to tenascin isoform 4 and fibronectin isoform 3, the morphogenesis formulation may further comprise at least one of:
Transforming growth factor beta induced protein ig-h3, with the amount of ig-h3 peptides being less than the amount of fibronectin peptides and more than the amount of tenascin peptides, a preferred ratio of 31:41 with respect to fibronectin peptides;
Plasminogen activator inhibitor 1, with the amount of plasminogen activator inhibitor 1 peptides being less than the amount of fibronectin peptides and more than the amount of tenascin peptides, a preferred ratio of 38:41 with respect to fibronectin peptides;
Amyloid beta A4 protein, with the amount of amyloid beta A4 peptides being than the amount of fibronectin peptides and more than the amount of tenascin peptides, a preferred ratio of 18:41 with respect to fibronectin peptides;
Glucose-6-phosphate isomerase, with the amount of isomerase peptides being less than the amount of fibronectin peptides and more than the amount of tenascin peptides, a preferred ratio of 19:41 with respect to fibronectin peptides;
Long isoform of serine protease inhibitor Kazal-type 5, with the amount of serine protease inhibitor being less than the amount of fibronectin peptides and more than the amount of tenascin peptides, a preferred ratio of 9:41 with respect to fibronectin peptides;
Cadherin-3, with the amount of cadherin-3 peptides being less than the amount of fibronectin peptides and more than the amount of tenascin peptides, a preferred ratio of 9:41 with respect to fibronectin peptides;
Pappalysin-1, with the amount of pappalysin-1 peptides being less than the amount of fibronectin peptides and less than the amount of tenascin peptides, a preferred ratio of 4:41 with respect to fibronectin peptides;
Insulin-like growth factor-binding protein 7, with the amount insulin-like growth factor-binding protein 7 peptides being less than the amount of fibronectin peptides and more than the amount of tenascin peptides, a preferred ratio of 24:41 with respect to fibronectin peptides;
Kallikrein-10, with the amount of kallikrein-10 peptides being less than the amount of fibronectin peptides and more than the amount of tenascin peptides, a preferred ratio of 22:41 with respect to fibronectin peptides;
Protocadherin fat 1, with the amount of protocadherin fat 1 peptides being less than the amount of fibronectin peptides and less than the amount of tenascin peptides, a preferred ratio of 1:41 with respect to fibronectin peptides;
Syntenin-1, with the amount of syntenin-1 peptides being less than the amount of fibronectin peptides and more than the amount of tenascin peptides, a preferred ratio of 14:41 with respect to fibronectin proteins;
Proliferation-associated protein 2G4, with the amount of proliferation-associated protein peptides being less than the amount of fibronectin peptides and more than the amount of tenascin peptides, a preferred ratio of 10:41 with respect to fibronectin peptides;
Protein CYR61, with the amount of protein CYR61 peptides being less than the amount of fibronectin peptides and more than the amount of tenascin peptides, a preferred ratio of 10:41 with respect to fibronectin peptides;
Keratinocyte proline-rich protein, with the amount of proline-rich protein peptides being less than the amount of fibronectin peptides and less than the amount of tenascin peptides, a preferred ratio of 6:41 with respect to fibronectin peptides;
Brain-specific serein protease 4, with the amount of serine protease peptides being less than the amount of fibronectin peptides, a preferred ratio of 8:41 with respect to fibronectin peptides;
Cadherin 13 isoform 4, with the amount of cadherin 13 peptides being less than the amount of fibronectin peptides and less than the amount of tenascin peptides, a preferred ratio of 4:41 with respect to fibronectin peptides;
Integrin alpha-2, with the amount of integrin alpha 2 peptides being less than the amount of fibronectin peptides and less than the amount of tenascin peptides, a preferred ratio of 2:41 with respect to fibronectin peptides;
Integrin beta-1, with the amount of beta-1 peptides being less than the amount of fibronectin peptides and less than the amount of tenascin peptides, a preferred ratio of 3:41 with respect to fibronectin peptides; and
Neural cell adhesion molecule L1 isoform 2, with the amount of adhesion molecule peptides being less than the amount of fibronectin peptides and less than the amount of tenascin peptides, a preferred ratio of 2:41 with respect to fibronectin peptides.

The stressed serum may comprise a sufficient amount of an extracellular matrix formulation comprising laminin subunit alpha 3.

In addition to laminin alpha subunit 3, the extracellular matrix formulation may further comprise at least one of:
Laminin subunit gamma 2, with the amount of gamma 2 peptides being greater than the amount of alpha 3 peptides, a preferred ratio of 42:30;
Laminin subunit beta 3, with the amount of beta 3 peptides being greater than the amount of alpha 3 peptides, a preferred ratio of 36:30;
Collagen alpha-1(I), with the amount of alpha-1(I) peptides being less than the amount of alpha 3 peptides, a preferred ratio of 11:30;
Collagen alpha-1(III), with the amount of alpha-1(III) peptides being less than the amount of alpha 3 peptides, a preferred ratio of 4:30;
Collagen alpha-1(VI), with the amount of alpha-(VI) peptides being less than the amount of alpha 3 peptides, a preferred ratio of 28:30;
Collagen alpha-2(I), with the amount of alpha-2(I) peptides being less than the amount of alpha 3 peptides, a preferred ratio of 15:30;
Collagen alpha-2(VI), with the amount of alpha-2(VI) peptides being less than the amount of alpha 3 peptides, a preferred ratio of 11:30;
Laminin subunit alpha 4, with the amount of alpha 4 peptides being less than the amount of alpha 3 peptides, a preferred ratio of 7:30;
Laminin subunit beta 1, with the amount of beta 1 peptides being less than the amount of alpha 3 peptides, a preferred ratio of 8:30;
Laminin subunit gamma 1, with the amount of gamma 1 peptides being less than the amount of alpha 3 peptides, a preferred ratio of 12:30;
Laminin 332; and
Laminin 411.

The stressed serum may comprise a sufficient amount of an immune promoting formulation comprising serapin B7, complement component C1s and complement component C3. The amount of C1s peptides may be less than the amount of complement C3 peptides and the amount of serapin B7 peptides may be more than the amount of the C3 peptides, with a preferred ratio of C1s to C3 peptides 12:14, and a preferred ratio of serapin peptides to C3 peptides of 18:14.

In addition to serapin B7, complement component C1s and complement component C3, the immune promoting formulation may further comprise at least one of:
Elafin, with the amount of elafin peptides being greater than the amount of serpin, C3 or C1s peptides, a preferred ratio of 74:14 with respect to C3 peptides;
Matrix metalloproteinase-9, with the amount of metalloproteinase peptides being less the amount of serpin, C3 or C1s peptides, a preferred ratio of 9:14 with respect to C3 peptides;
Stromelysin-2, with the amount of stromelysin-2 peptides being less the amount of serpin, C3 or C1s peptides, a preferred ratio of 9:14 with respect to C3 peptides;
HLA class I histocompatibility antigen, Cw-6 alpha chain, with the amount Cw-6 alpha chain peptides being less the amount of serpin, C3 or C1s peptides, a preferred ratio of 6:14 with respect to C3 peptides;
Quinone oxidoreductase PIG3, with the amount PIG3 peptides being less the amount of serpin, C3 or C1s peptides, a preferred ratio of 10:14 with respect to C3 peptides;
Superoxide dismutase, with the amount of dismutase peptides being greater than the amount of C1s peptides and less than the serapin peptides, a preferred ratio of 1:1 with respect to C3 peptides;
Metallothionein-2, with the amount of metallothionein peptides being more than the amount of C1s, C3 or serapin peptides, a preferred ratio of 42:14 with respect to C3 peptides;
Alpha-1 antichymotrypsin, with the amount of anitchymotrypsin peptides being less the amount of serpin, C3 or C1s peptides, a preferred ratio of 6:14 with respect to C3 peptides;
Interleukin-1 receptor-like 1, with the amount of interleukin-1 receptor-like peptides being less the amount of serpin, C3 or C1s peptides, a preferred ratio of 4:14 with respect to C3 peptides; and Neutrophil chemotactic agents.

The Neutrophil chemotactic agents may comprise at least one of:
Interleukin-6, with the amount of interleukin-6 peptides being more the amount of serpin, C3 or C1s peptides, a preferred ratio of 23:14 with respect to C3 peptides;
Growth regulated alpha protein, with the amount of alpha protein peptides being more the amount of serpin, C3 or C1s peptides, a preferred ratio of 32:14 with respect to C3 peptides;
Protein S100-A8, with the amount S100-A8 peptides being more the amount of serpin, C3 or C1s peptides, a preferred ratio of 30:14 with respect to C3 peptides;
Interleukin-8, with the amount of interleukin-8 peptides being more the amount of serpin, C3 or C1s peptides, a preferred ratio of 26:14 with respect to C3 peptides; and
C—X—C motif chemokine 5, the amount of chemokine peptides being more the amount of serpin, C3 or C1s peptides, a preferred ratio of 19:14 with respect to C3 peptides.

The stressed serum may comprise a sufficient amount of an extracellular matrix modification formulation comprising interstitial collagenase and stomelysin-1. The amount of stomelysin-1 peptides may be less than the amount of collagenase peptides, with a preferred ratio of 46:72.

In addition to interstitial collagenase and stomelysin-1, the extracellular matrix modification formulation may further comprise at least one of:
Cathepsin L2, with that amount of cathepsin peptides less than the amount of stomelysin peptides and less than the amount of collagenase peptides, a preferred ratio of 37:72 with respect to collagenase peptides;
Latent transforming growth factor beta-binding protein 2, with the amount of latent transforming growth factor beta-binding protein peptides less than the amount of stomelysin peptides and less than the amount of collagenase peptides, a preferred ratio of 6:72 with respect to collagenase peptides;
Aminopeptidase N, with the amount of aminopeptidase peptides peptides less than the amount of stomelysin peptides and less than the amount of collagenase peptides, a preferred ratio of 6:72 with respect to collagenase peptides;
Decorin, with the amount of decorin peptides less than the amount of stomelysin peptides and less than the amount of collagenase peptides, a preferred ratio of 27:72 with respect to collagenase peptides;
Urokinase-type plasminogen activator, with the amount of plasminogen activators peptides less than the amount of stomelysin peptides and less than the amount of collagenase peptides, a preferred ratio of 20:72 with respect to collagenase peptides;
Lumican, with the amount lumican peptides less than the amount of stomelysin peptides and less than the amount of collagenase peptides, a preferred ratio of 21:72 with respect to collagenase peptides;
Cystatin-M, with the amount of cystatin peptides less than the amount of stomelysin peptides and less than the amount of collagenase peptides, a preferred ratio of 30:72 with respect to collagenase peptides;
Lysyl oxidase homolog 2, with the amount of homolog peptides less than the amount of stomelysin peptides and less than the amount of collagenase peptides, a preferred ratio of 5:72 with respect to collagenase;
Cystatin-C, the amount of cystatin peptides less than the amount of stomelysin peptides and less than the amount of collagenase peptides, a preferred ratio of 25:72 with respect to collagenase peptides;
Protein-lysine 6-oxidase, the amount of oxidase peptides less than the amount of stomelysin peptides and less than the amount of collagenase peptides, a preferred ratio of 8:72 with respect to collagenase peptides;
Tissue factor pathway inhibitor 2, the amount of inhibitor peptides less than the amount of stomelysin peptides and less than the amount of collagenase peptides, a preferred ratio of 14:72 with respect to colleganase; and
Procollagen-lysine, 2-oxoglutarate 5-dioxgenase isoform 2, the amount of dioxgenase peptides less than the amount of stomelysin peptides and less than the amount of collagenase peptides, a preferred ratio of 3:72 with respect to colleganase.

The stressed serum may comprise a sufficient amount of cytoskeleton formulation comprising translationally-controlled tumor protein.

In addition to translationally-controlled tumor protein, the cytoskeleton formulation may further comprise at least one of:
Filamin A, with the amount of filamin peptides being less than amount translationally-controlled tumor protein peptides, a preferred ratio of 13:44;
Alpha-actin-1, with the amount of alpha-actin peptides being less than amount translationally-controlled tumor protein peptides, a preferred ratio of 31:44;
Microtubule associated protein 4, with the amount of microtubule associated protein peptides being less than amount translationally-controlled tumor protein peptides, a preferred ratio of 19:44;
Moesin, with the amount of moesin peptides being less than amount translationally-controlled tumor protein peptides, a preferred ratio of 35:44;
Vinculin, with the amount of vinculin peptides being less than amount translationally-controlled tumor protein peptides, a preferred ratio of 11:44;
Involucrin, with the amount involucrin peptides being less than amount translationally-controlled tumor protein peptides, a preferred ratio of 18:44;
Gelsolin isoform 2, with the amount gelsolin peptides being less than amount translationally-controlled tumor protein peptides, a preferred ratio of 10:44;
PDZ and LIM domain protein 1, with amount of PDZ and LIM domain protein peptides being less than amount translationally-controlled tumor protein peptides, a preferred ratio of 20:44;
Caldesmon isoform 5, with the amount of caldesmon peptides being less than amount translationally-controlled tumor protein peptides, a preferred ratio of 13:44;
LIM domain and actin-binding protein 1, with the amount of LIM domain and actin-binding protein peptides being less than amount translationally-controlled tumor protein peptides, a preferred ratio of 7:44;
Myosin regulatory light chain 12B, with the amount light chain peptides being less than amount translationally-controlled tumor protein peptides, a preferred ratio of 21:44;
Small proline rich protein 3, with the amount protein 3 peptides being less than amount translationally-controlled tumor protein peptides, a preferred ratio of 22:44;
Smooth muscle isoform of myosin light polypeptide 6, with the amount of myosin peptides being less than amount translationally-controlled tumor protein peptides, a preferred ratio of 23:44;
Jupiter microtubule associated homolog 1, with the amount of Jupiter peptides being less than amount translationally-controlled tumor protein peptides, a preferred ratio of 21:44;
Small proline rich protein 2A, with the amount of protein 2A peptides being less than amount translationally-controlled tumor protein peptides, a preferred ratio of 45:44; and
Myotrophin, with the amount of myotrophin peptides being less than amount translationally-controlled tumor protein peptides, a preferred ratio of 22:44.

The stressed serum may further comprise at least one of:
A chaperone formulation comprising at least one of 78 kDa glucose-regulated protein and heat shock 70 kDA protein to assist protein folding and/or assembly;
Nidogen-1 to maintain the basement membrane;
Serum albumin; and
A metabolism formulation comprising at least one of pyruvate kinase, phospholipid transfer protein,

The stressed serum collected may be combined with cells to be implanted before cryopreservation, during thawing, prior to implantation and/or after implantation. For instance, the stressed serum may be incorporated into a cryopreservation solution. The stressed serum may also be combined with cells by thawing cryopreserved cells in a solution comprising a sufficient amount of the stressed. The stressed serum may also be combined with the cells by incorporating a sufficient amount of the stressed serum into a graft and/or matrix containing the cells. The stressed serum may also be combined with the cells by including a sufficient amount of the stressed serum within an injection solution, spray and/or other delivery vehicle utilized to implant the stem cells. Cells may also be combined with the stressed serum by placing a matrix, such as, but not limited to, a hydrogel and/or bandage, containing a sufficient amount of the stressed serum over, under, adjacent and/or in proximity to implanted cells. Applying a solution, such as, but limited to a spray or injection, containing the stressed serum onto and/or adjacent the implanted cells may also combine the stem cells with the stressed serum.

It is thus understood from the description herein that aspects of the invention provide new cryopreserved cells comprising: frozen cells; a cryopreservation solution; and a sufficient amount of a stressed serum. In embodiments, the stressed serum is obtained by: seeding a first surface with a first culture of cells; growing the first culture to a monolayer of less than 100% confluence in the presence of a first growth medium as to provide at least one cell free area on the first surface, the first grown medium comprising nutrients and at least one growth factor, wherein the first growth medium promotes proliferation of the first culture; after growing the first culture to less than 100% confluence, replacing the growth medium with a collection medium, the collection medium lacking at least one of the growth factors of the growth medium; maintaining the first culture for a period of time in the presence of the collection medium to produce a conditioned medium; and collecting at least a portion of the conditioned medium after the period of time, said conditioned medium comprising the stressed serum. In embodiments, the obtaining the stressed serum further comprises: seeding a second culture onto the at least one cell free area of the first surface, the second culture comprising cells different from the first culture; growing the first and second culture to less than 100% confluence in the presence the second growth medium, the second growth medium comprising of nutrients and at least one growth factor, wherein the second growth medium promotes proliferation of the second culture; after growing the first and second cultures to less than 100% confluence, replacing the second growth medium with a collection medium, the collection medium lacking at least one of the growth factors of the second growth medium; maintaining the first and second cultures for a period of time in the presence of the collection medium to produce a conditioned medium; and collecting at least a portion of the conditioned medium after the period of time, said conditioned medium comprising the stressed serum. In embodiments, the stressed serum comprises a sufficient amount of at least one of amount of an angio-modifying formulation, a morphogenesis formulation, an extracellular matrix formulation, an extracellular matrix modification formulation, an immune promoting formulation and a cytoskeleton formulation.

It is thus understood from the description herein that aspects of the invention provide a new graft comprising: a biocompatible matrix; cells within biocompatible matrix; and a sufficient amount of stressed serum within the biocompatible matrix. In embodiments, the stressed serum is obtained by: seeding a first surface with a first culture of cells; growing the first culture to a monolayer of less than 100% confluence in the presence of a first growth medium as to provide at least one cell free area on the first surface, the first grown medium comprising nutrients and at least one growth factor, wherein the first growth medium promotes proliferation of the first culture; after growing the first culture to less than 100% confluence, replacing the growth medium with a collection medium, the collection medium lacking at least one of the growth factors of the growth medium; maintaining the first culture for a period of time in the presence of the collection medium to produce a conditioned medium; and collecting at least a portion of the conditioned medium after the period of time, said conditioned medium comprising the stressed serum. In embodiments, the obtaining the stressed serum further comprises: seeding a second culture onto the at least one cell free area of the first surface, the second culture comprising cells different from the first culture; growing the first and second culture to less than 100% confluence in the presence the second growth medium, the second growth medium comprising of nutrients and at least one growth factor, wherein the second growth medium promotes proliferation of the second culture; after growing the first and second cultures to less than 100% confluence, replacing the second growth medium with a collection medium, the collection medium lacking at least one of the growth factors of the second growth medium; maintaining the first and second cultures for a period of time in the presence of the collection medium to produce a conditioned medium; and collecting at least a portion of the conditioned medium after the period of time, said conditioned medium comprising the stressed serum. In embodiments, the stressed serum comprises a sufficient amount of at least one of amount of an angio-modifying formulation, a morphogenesis formulation, an extracellular matrix formulation, an extracellular matrix modification formulation, an immune promoting formulation and a cytoskeleton formulation.

It is thus understood from the description herein that aspects of the invention provide a new method of recovering cryopreserved cells, comprising thawing the cells in a stressed serum solution. In embodiments, the stressed serum is obtained by: seeding a first surface with a first culture of cells; growing the first culture to a monolayer of less than 100% confluence in the presence of a first growth medium as to provide at least one cell free area on the first surface, the first grown medium comprising nutrients and at least one growth factor, wherein the first growth medium promotes proliferation of the first culture; after growing the first culture to less than 100% confluence, replacing the growth medium with a collection medium, the collection medium lacking at least one of the growth factors of the growth medium; maintaining the first culture for a period of time in the presence of the collection medium to produce a conditioned medium; and collecting at least a portion of the conditioned medium after the period of time, said conditioned medium comprising the stressed serum. In embodiments, the obtaining the stressed serum further comprises: seeding a second culture onto the at least one cell free area of the first surface, the second culture comprising cells different from the first culture; growing the first and second culture to less than 100% confluence in the presence the second growth medium, the second growth medium comprising of nutrients and at least one growth factor, wherein the second growth medium promotes proliferation of the second culture; after growing the first and second cultures to less than 100% confluence, replacing the second growth medium with a collection medium, the collection medium lacking at least one of the growth factors of the second growth medium; maintaining the first and second cultures for a period of time in the presence of the collection medium to produce a conditioned medium; and collecting at least a portion of the conditioned medium after the period of time, said conditioned medium comprising the stressed serum. In embodiments, the stressed serum comprises a sufficient amount of at least one of amount of an angio-modifying formulation, a morphogenesis formulation, an extracellular matrix formulation, an extracellular matrix modification formulation, an immune promoting formulation and a cytoskeleton formulation.

It is thus understood from the description herein that aspects of the invention provide a new method of producing a stressed serum improving replenishment of cells, comprising: seeding a first surface with a first culture of cells; growing the first culture to a monolayer of less than 100% confluence in the presence of a first growth medium as to provide at least one cell free area on the first surface, the first grown medium comprising nutrients and at least one growth factor, wherein the first growth medium promotes proliferation of the first culture; after growing the first culture to less than 100% confluence, replacing the growth medium with a collection medium, the collection medium lacking at least one of the growth factors of the growth medium; maintaining the first culture for a period of time in the presence of the collection medium to produce a conditioned medium; and collecting at least a portion of the conditioned medium after the period of time, said conditioned medium comprising the stressed serum. In embodiments, the first culture of cells comprises at least one of stem cells, cardiac cells, fibroblasts, keratinocytes and hepatic cells. In embodiments, the method further comprising: seeding a second culture onto the at least one cell free area of the first surface, the second culture comprising cells different from the first culture; growing the first and second culture to less than 100% confluence in the presence the second growth medium, the second growth medium comprising of nutrients and at least one growth factor, wherein the second growth medium promotes proliferation of the second culture; after growing the first and second cultures to less than 100% confluence, replacing the second growth medium with a collection medium, the collection medium lacking at least one of the growth factors of the second growth medium; maintaining the first and second cultures for a period of time in the presence of the collection medium to produce a conditioned medium; and collecting at least a portion of the conditioned medium after the period of time, said conditioned medium comprising the stressed serum, wherein at least one of the first culture and second cultures comprise stem cells.

It is thus understood from the description herein that aspects of the invention provide a new method of treating replenishing cells within a tissue, comprising administering to a tissue to be treated stressed serum comprising a sufficient amount of at least one of amount of an angio-modifying formulation, a morphogenesis formulation, an extracellular matrix formulation, an extracellular matrix modification formulation, an immune promoting formulation and a cytoskeleton formulation.

It is thus understood from the description herein that aspects of the invention provide a new method of implanting cells, comprising: combining cells with a stressed serum, the stressed serum comprising a sufficient amount of at least one of amount of an angio-modifying formulation, a morphogenesis formulation, an extracellular matrix formulation, an extracellular matrix modification formulation, an immune promoting formulation and a cytoskeleton formulation; and implanting the cells into a patient. In embodiments, the stems are frozen and the stressed serum is combined during thawing. In embodiments, the method further comprising combining the cells and stressed serum with a cryopreservation solution; and freezing the combination of cells, stressed serum and cryopreservation solution.

It is thus understood from the description herein that aspects of the invention provide a new therapeutic matrix, comprising: a biocompatible matrix; and a sufficient amount of a stressed serum within the biocompatible matrix. In embodiments, the stressed serum is obtained by: seeding a first surface with a first culture of cells; growing the first culture to a monolayer of less than 100% confluence in the presence of a first growth medium as to provide at least one cell free area on the first surface, the first grown medium comprising nutrients and at least one growth factor, wherein the first growth medium promotes proliferation of the first culture; after growing the first culture to less than 100% confluence, replacing the growth medium with a collection medium, the collection medium lacking at least one of the growth factors of the growth medium; maintaining the first culture for a period of time in the presence of the collection medium to produce a conditioned medium; and collecting at least a portion of the conditioned medium after the period of time, said conditioned medium comprising the stressed serum. In embodiments, the obtaining the stressed serum further comprises: seeding a second culture onto the at least one cell free area of the first surface, the second culture comprising cells different from the first culture; growing the first and second culture to less than 100% confluence in the presence the second growth medium, the second growth medium comprising of nutrients and at least one growth factor, wherein the second growth medium promotes proliferation of the second culture; after growing the first and second cultures to less than 100% confluence, replacing the second growth medium with a collection medium, the collection medium lacking at least one of the growth factors of the second growth medium; maintaining the first and second cultures for a period of time in the presence of the collection medium to produce a conditioned medium; and collecting at least a portion of the conditioned medium after the period of time, said conditioned medium comprising the stressed serum. In embodiments, the stressed serum comprises a sufficient amount of at least one of amount of an angio-modifying formulation, a morphogenesis formulation, an extracellular matrix formulation, an extracellular matrix modification formulation, an immune promoting formulation and a cytoskeleton formulation.

While the present invention has been described herein with respect to the exemplary embodiments, it will become apparent to one of ordinary skill in the art that many modifications, improvements and sub-combinations of the various embodiments, adaptations and variations can be made to the invention without departing from the spirit and scope thereof. Accordingly, the presented embodiment should not be construed as limiting the scope of this disclosure or the accompanying claims.

Furthermore, it should be appreciated that “first” and “second” as used in claims is merely to reference that one (first) precedes another (second) and/or to distinguish similar components from one another. It should also be appreciated that though examples presented above may have included two cultures, this was solely for purposes of illustration and in no way intended to limit the scope of this disclosure or the claims. As such, a “first culture” may be a first, second, third, etc. culture. Likewise, the “second culture” may be any culture added subsequent to the “first culture”. Accordingly, if the “first culture” is the third culture added, then the “second culture” may be a fourth, fifth, sixth, etc. culture.

What is claimed:

1. Cryopreserved cells, comprising:
frozen cells;
a cryopreservation solution; and
a sufficient amount of a stressed serum.

2. The cryopreserved cells of claim 1, wherein the stressed serum is obtained by:
seeding a first surface with a first culture of cells;
growing the first culture to a monolayer of less than 100% confluence in the presence of a first growth medium as to provide at least one cell free area on the first surface, the first grown medium comprising nutrients and at least one growth factor, wherein the first growth medium promotes proliferation of the first culture;
after growing the first culture to less than 100% confluence, replacing the growth medium with a collection medium, the collection medium lacking at least one of the growth factors of the growth medium;
maintaining the first culture for a period of time in the presence of the collection medium to produce a conditioned medium; and
collecting at least a portion of the conditioned medium after the period of time, said conditioned medium comprising the stressed serum.

3. The cryopreserved cells of claim 2, wherein obtaining the stressed serum further comprises:
seeding a second culture onto the at least one cell free area of the first surface, the second culture comprising cells different from the first culture;
growing the first and second culture to less than 100% confluence in the presence the second growth medium, the second growth medium comprising of nutrients and at least one growth factor, wherein the second growth medium promotes proliferation of the second culture;
after growing the first and second cultures to less than 100% confluence, replacing the second growth medium with a collection medium, the collection medium lacking at least one of the growth factors of the second growth medium;
maintaining the first and second cultures for a period of time in the presence of the collection medium to produce a conditioned medium; and
collecting at least a portion of the conditioned medium after the period of time, said conditioned medium comprising the stressed serum.

4. The cryopreserved cells of claim 1, wherein the stressed serum comprises a sufficient amount of at least one of amount of an angio-modifying formulation, a morphogenesis formulation, an extracellular matrix formulation, an extracellular matrix modification formulation, an immune promoting formulation and a cytoskeleton formulation.

5. A graft comprising:
a biocompatible matrix;
cells within biocompatible matrix; and
a sufficient amount of stressed serum within the biocompatible matrix.

6. The graft of claim 5, wherein the stressed serum is obtained by:
seeding a first surface with a first culture of cells;
growing the first culture to a monolayer of less than 100% confluence in the presence of a first growth medium as to provide at least one cell free area on the first surface, the first grown medium comprising nutrients and at least one growth factor, wherein the first growth medium promotes proliferation of the first culture;
after growing the first culture to less than 100% confluence, replacing the growth medium with a collection medium, the collection medium lacking at least one of the growth factors of the growth medium;
maintaining the first culture for a period of time in the presence of the collection medium to produce a conditioned medium; and
collecting at least a portion of the conditioned medium after the period of time, said conditioned medium comprising the stressed serum.

7. The graft of claim 5, wherein obtaining the stressed serum further comprises:
seeding a second culture onto the at least one cell free area of the first surface, the second culture comprising cells different from the first culture;
growing the first and second culture to less than 100% confluence in the presence the second growth medium, the second growth medium comprising of nutrients and at least one growth factor, wherein the second growth medium promotes proliferation of the second culture;
after growing the first and second cultures to less than 100% confluence, replacing the second growth medium with a collection medium, the collection medium lacking at least one of the growth factors of the second growth medium;
maintaining the first and second cultures for a period of time in the presence of the collection medium to produce a conditioned medium; and
collecting at least a portion of the conditioned medium after the period of time, said conditioned medium comprising the stressed serum.

8. The graft of claim 5, wherein the stressed serum comprises a sufficient amount of at least one of amount of an angio-modifying formulation, a morphogenesis formulation, an extracellular matrix formulation, an extracellular matrix modification formulation, an immune promoting formulation and a cytoskeleton formulation.

9. A method of recovering cryopreserved cells, comprising thawing the cells in a stressed serum solution.

10. The method of claim 9, wherein the stressed serum is obtained by:
seeding a first surface with a first culture of cells;
growing the first culture to a monolayer of less than 100% confluence in the presence of a first growth medium as to provide at least one cell free area on the first surface, the first grown medium comprising nutrients and at least one growth factor, wherein the first growth medium promotes proliferation of the first culture;
after growing the first culture to less than 100% confluence, replacing the growth medium with a collection medium, the collection medium lacking at least one of the growth factors of the growth medium;
maintaining the first culture for a period of time in the presence of the collection medium to produce a conditioned medium; and
collecting at least a portion of the conditioned medium after the period of time, said conditioned medium comprising the stressed serum.

11. The method of claim 9, wherein obtaining the stressed serum further comprises:
seeding a second culture onto the at least one cell free area of the first surface, the second culture comprising cells different from the first culture;
growing the first and second culture to less than 100% confluence in the presence the second growth medium, the second growth medium comprising of nutrients and at least one growth factor, wherein the second growth medium promotes proliferation of the second culture;
after growing the first and second cultures to less than 100% confluence, replacing the second growth medium with a collection medium, the collection medium lacking at least one of the growth factors of the second growth medium;
maintaining the first and second cultures for a period of time in the presence of the collection medium to produce a conditioned medium; and
collecting at least a portion of the conditioned medium after the period of time, said conditioned medium comprising the stressed serum.

12. The method of claim 9, wherein the stressed serum comprises a sufficient amount of at least one of amount of an angio-modifying formulation, a morphogenesis formulation, an extracellular matrix formulation, an extracellular matrix modification formulation, an immune promoting formulation and a cytoskeleton formulation.
